(19) United States
(12) Patent Application Publication
(10) Pub. No.: US 2003/0181869 A1
(43) Pub. Date: Sep. 25, 2003

(54) Needle assembly

(75) Inventors: Kirk D. Swenson, North Caldwell, NJ (US); Volker Niermann, Bound Brook, NJ (US)

Correspondence Address: Kirk M. Miles, Esq., WEBB ZIESENHEIM LOGSDON ORKIN & HANSON, P.C., 700 Koppers Building, 436 Seventh Avenue, Pittsburgh, PA, 15219-1818, US

(73) Assignee: Becton, Dickinson and Company

(21) Appl. No.: 10/391,984

(22) Filed: Mar. 19, 2003

Related U.S. Application Data
(60) Provisional application No. 60/365,922, filed on Mar. 20, 2002.

(51) Int. Cl. A61M 5/32

(52) U.S. Cl. 604/263

(57) ABSTRACT

The shieldable needle assembly includes a needle cannula and a hub member supporting a proximal end of the needle cannula. A shield member is aligned with the hub member and extends co-axially about the needle cannula. The shield member is movable between a retracted position in which a puncture tip of the needle cannula is exposed, and an extended position covering the puncture tip. A pair of extendable members is connected between the hub and the shield members. The extendable members each include a pair of folding legs. A tension spring is connected between the extendable members. The hub member is movable in an axial direction away from the shield member.

### CROSS REFERENCE TO RELATED APPLICATIONS

This application claims the benefit of U.S. Provisional Application No. 60/365,922 filed on Mar. 20, 2002.

### BACKGROUND OF THE INVENTION

1. Field of the Invention

The present invention relates to blood collection sets for safe and convenient handling of needles. More particularly, the present invention relates to a blood collection set including a needle assembly having a low profile, forward moving safety shield for protection from a used needle tip.

2. Description of Related Art

Disposable medical devices having medical needles are used for administering medication or withdrawing fluid from the body of a patient. Such disposable medical devices typically include blood collecting needles, fluid handling needles, and assemblies thereof. Current medical practice requires that fluid containers and needle assemblies used in such devices be inexpensive and readily disposable. Consequently, existing blood collection devices typically employ some form of durable, reusable holder on which detachable and disposable medical needles and fluid collection tubes may be mounted. A blood collection device of this nature may be assembled prior to use and then disassembled after use. Thus, these blood collection devices allow repeated use of a relatively expensive holder upon replacement of relatively inexpensive medical needles and/or fluid collection tubes. In addition to reducing the cost of collecting blood specimens, these blood collection devices help minimize the production of hazardous waste material.

A blood collection device or intravenous (IV) infusion device typically includes a needle cannula having a proximal end, a pointed distal end, and a lumen extending therebetween. The proximal end of the needle cannula is securely mounted in a plastic hub defining a central passage that communicates with the lumen extending through the needle cannula. A thin, flexible thermoplastic tube is connected to the hub and communicates with the lumen of the needle cannula. The end of the plastic tube remote from the needle cannula may include a fixture for connecting the needle cannula to a blood collection tube or other receptacle. The specific construction of the fixture will depend upon the characteristics of the receptacle to which the fixture is to be connected.

In order to reduce the risk of incurring an accidental needle-stick wound, protection of used needle cannulas becomes important. With concern about infection and transmission of diseases, methods and devices to enclose or cover the used needle cannula have become very important and in great demand in the medical field. For example, needle assemblies commonly employ a safety shield that can be moved into shielding engagement with a used needle cannula to minimize risk of an accidental needle-stick.

Some needle safety shields are referred to as “tip guards” and include a small rigid guard that may be telescoped along the length of the needle cannula and extended over the pointed distal end of the needle cannula for protection. Such conventional tip guards may include some form of tether for limiting the travel of the tip guard to the length of the needle cannula. An example of the foregoing is disclosed by U.S. Pat. No. 5,176,655 to McCormick et al. The McCormick et al. patent discloses the use of flexible loop-like straps for limiting the distal movement of a tip guard.

Needle shields that incorporate movable tip guards are typically manually actuated. For example, U.S. Pat. Nos. Re 36,447 and Re 36,398, both to Byrne et al., disclose a safety device for a hypodermic needle that includes a plastic sheath, which is used to cover the puncture tip of the needle. The plastic sheath incorporates a thumb guard, which the user of the safety device may grasp to move the plastic sheath to a position covering the puncture tip of the needle. U.S. Pat. No. 5,951,525 to Thorne et al. discloses a manually operated safety needle apparatus that includes two pairs of opposed legs adapted to move the tip guard of the apparatus to a position covering the used needle cannula. U.S. Pat. Nos. 5,562,637 and 5,562,636, both to Utterburg, disclose a rectangular needle protector sheath for use with a needle cannula that may be extended over the needle cannula after its use. Other prior art devices, such as those disclosed by U.S. Pat. Nos. 5,290,264 to Utterberg and 5,192,275 to Bums, provide “grippable” members attached to the tip guards to facilitate moving the tip guards to a position covering the puncture tip of a needle cannula. In addition to providing gripping members for moving the tip guards, prior art devices in this area often include flexible wings, which are used as means for securing the needle assemblies to the body of a patient during a medical procedure. Examples of “winged” needle assemblies may found in U.S. Pat. Nos. 5,120,320 to Fayngold; and 5,154,699; 5,088,982; and 5,085,639 all to Ryan. Other prior art in this area includes U.S. Pat. Nos. 5,266,072 and 5,112,311, both to Utterberg et al., which also disclose guarded winged needle assemblies.

Conventional tip guards, such as those discussed hereinabove, often include a structure that lockingly engages over the pointed distal end of the used needle cannula to prevent a re-exposure of the needle cannula. The structure for preventing the re-exposure of the needle cannula may include a metallic spring clip or a transverse wall formed integrally with one end of the tip guard. An example of a metallic spring clip is disclosed by the McCormick et al. patent discussed previously.

Conventional tip guards, such as those discussed hereinabove, often further require extensive mechanics for positioning the tip guard over the needle cannula. This results in complex arrangements that are costly to manufacture and assemble. Additionally, operation of the needle assemblies to move the tip guard into the proper position over the pointed distal end of the needle cannula requires substantial manual manipulation by the user of the device, exposing the user to potential needle-stick wounds.

In view of the foregoing, a need exists for a blood collection set including a shieldable needle assembly that achieves secure and effective shielding of a used needle cannula, which is simple and inexpensive to manufacture and easy to operate.

### SUMMARY OF THE INVENTION

The present invention is directed to a blood collection set incorporating a shield needle assembly. The blood collection set generally includes a fixture for connecting the blood collection set to a receptacle, a flexible tube, and the shieldable needle assembly. The flexible tube has opposed first and seconds ends, with the first end of the flexible tube connected to the fixture. The needle assembly used in the blood collection set includes a needle cannula having a proximal end and a distal end with a puncture tip. The proximal end of the needle cannula is connected to a hub member, which supports the proximal end of the needle cannula. The distal end of the needle cannula projects outward from the hub member. The needle cannula defines a lumen in fluid communication with the flexible tube and the fixture. The needle assembly includes a shield member having a housing defining a central bore. The shield member is in axial alignment with the hub member and extends co-axially about the needle cannula. The shield member and the hubmaker are adapted for relative axial movement with respect to each other in opposing axial directions between a first position in which the puncture tip of the needle cannula is exposed and a second position in which the shield member covers the puncture tip of the needle cannula. The needle assembly further includes a pair of extendable members connecting the hub member and the shield member. The extendable members each include a pair of folding legs connected by a hinged knee joint. One leg of the pair of legs for each extendable member is connected hingedly to the hub member at a hub hinge and the other leg of the pair of legs for each extendable member is connected hingedly to the shield member. A tension spring is operatively connected between the extendable members. The hub hinge is located in front of the tension spring when the shield member is in the retracted position. The hub member is movable in an axial direction away from a proximal end of the shield member. When the legs connected to the hub member for each extendable member are pivoted to a position forming an angle of greater than 90° with the axis of the needle cannula, the shield member and the hub member are biased in opposing axial directions to the second position by the tension spring.

The shield member may include a pair of butterfly wings extending laterally from opposing sides of the housing of the shield member. In addition, the housing of the shield member may further include a dorsal wing located between the butterfly wings.

The shield member may further include a tip guard for protectively surrounding the puncture tip of the needle cannula when the shield member is moved to the second position. The tip guard may comprise a tip guard housing and a spring clip connected to the tip guard housing. The spring clip may be biased against the needle cannula when the shield member is in the first position and resiliently extends over the puncture tip of the needle cannula when the shield member is in the second position.

Alternatively, the tip guard may be externally attached to the housing of the shield member. The externally secured tip guard may comprise a spring leg extending axially along the housing, a pair of clip legs securing the tip guard to the housing, and a locking plate resiliently biased against the needle cannula when the shield member is in the first position. The locking plate is configured to resiliently extend over a distal opening to the central bore of the housing when the shield member is moved to the second position.

The knee joint for each extendable member may further include a fingerplate to enable the user to manipulate the needle assembly. The needle assembly may further include a locking assembly extending from the fingerplate of at least one of the extendable members toward the needle cannula and configured to engage the needle cannula such that when the shield member is moved to the second position by the tension spring, the locking assembly automatically engages onto the needle cannula to secure the shield member in the second position. The locking assembly may be comprised of a pair of opposed locking members defining a recess therebetween for receiving the needle cannula. The locking members are preferably made of a resiliently flexible material such that the needle cannula is able to separate the locking members under force provided by the tension spring.

The locking assembly may be configured to connect the knee joints of the extendable members, and be comprised of a locking member having a plurality of ratchet teeth. The ratchet teeth of the locking member may be configured to permit unidirectional movement of the knee joints of the extendable members toward one another. The locking member may be connected between the fingerplates of the extendable members.

The hub member may be adapted for connection to the flexible tube of the blood collection set.

A removable protective sleeve may be positioned over the distal end of the needle cannula.

The present invention further includes a method of automatically placing a shieldable needle assembly into a safety state in which a puncture tip of a needle cannula is covered by the needle assembly. The method generally comprises the steps of providing the shieldable needle assembly as described hereinabove, using the shieldable needle assembly in a medical procedure, and moving the hub member in an axial direction away from a proximal end of the shield member until the legs connected to the hub member for each extendable member are pivoted to a position forming an angle of greater than 90° with the axis of the needle cannula such that the shield member and the hub member are biased in opposing axial directions to the second position by the tension spring. The method may further include the step of automatically engaging the locking members of the locking assembly onto the needle cannula when the shield member is biased to the second position by the tension spring.

Further details and advantages of the present invention will become apparent from the following detailed description read in conjunction with the drawings.

### BRIEF DESCRIPTION OF THE DRAWINGS

FIG. 1 is a perspective view of a blood collection set including a shieldable needle assembly in accordance with the present invention and showing the needle assembly in a retracted position;

FIG. 2 is a cross-sectional view taken along line II-II in FIG. 1;

FIG. 3 is a cross-sectional view taken along line III-III in FIG. 1;

FIG. 4 is a perspective view of the shieldable needle assembly of FIG. 1 shown in a partially extended position;

FIG. 5 is a perspective view of the shieldable needle assembly of FIG. 1 shown in a further extended position;

FIG. 6 is a perspective view of the shieldable needle assembly of FIG. 1 shown in a fully extended position;

FIG. 7 is a side view of a tip guard used in the shieldable needle assembly of FIG. 1 shown prior to activation;

FIG. 8 is a side view of the tip guard of FIG. 7 shown in an activated, shielding position;

FIG. 9 is a perspective view of the shieldable needle assembly of FIG. 1 having a modified tip guard in accordance with the present invention;

FIG. 10 is a cross-sectional view taken along lines X-X in FIG. 9;

FIG. 11 is a cross-sectional view of the modified tip guard of FIG. 9 showing the tip guard covering a needle cannula of the shieldable needle assembly; and

FIG. 12 is a perspective view of the blood collection set and shieldable needle assembly is accordance with a second embodiment of the present invention.

### DETAILED DESCRIPTION

Referring to the drawings in which like reference characters refer to like parts throughout the several views thereof, the present invention is generally described in terms of a blood collection set and related features, and encompasses such a blood collection set as well as a shieldable needle assembly for use in such a blood collection set.

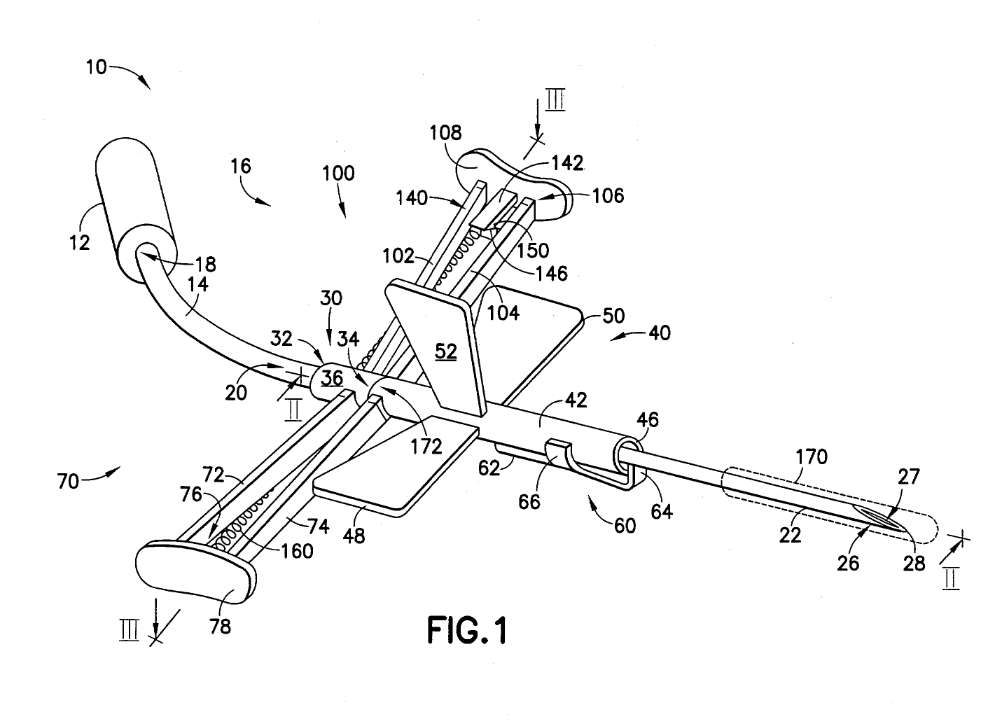

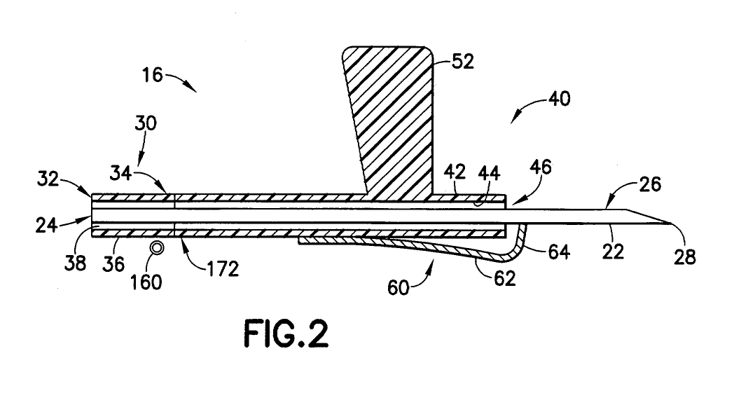

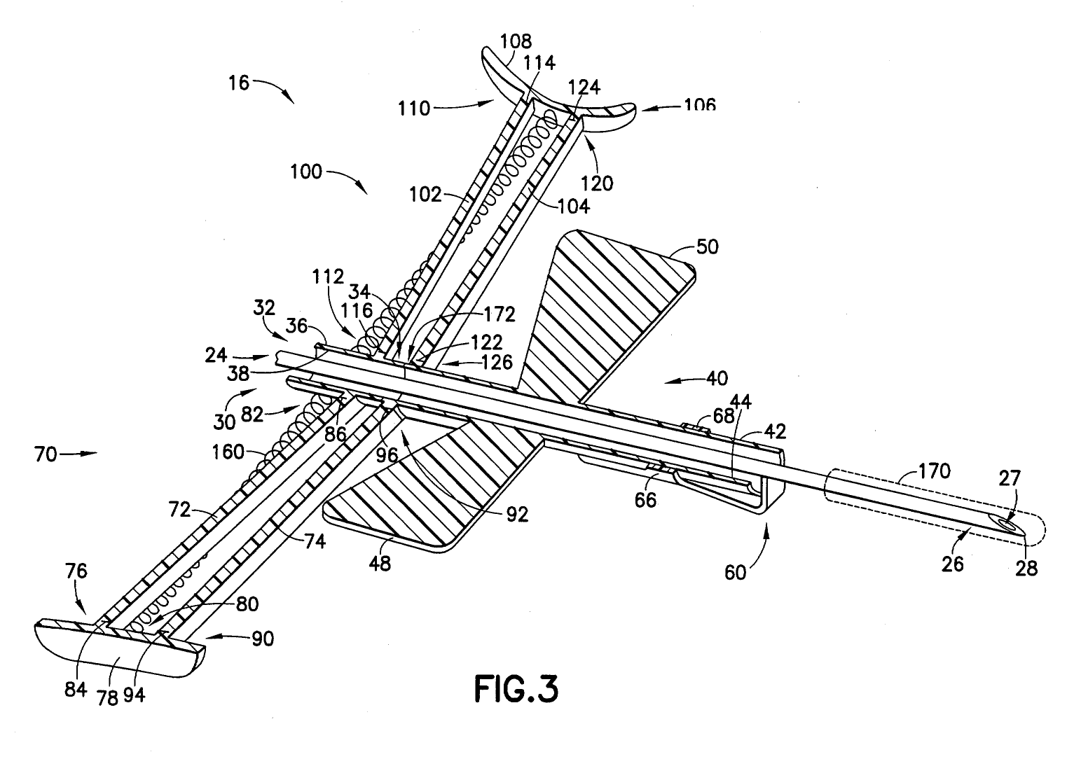

Referring to FIGS. 1-6, a blood collection set 10 in accordance with the present invention includes a fixture 12 for connecting the blood collection set 10 to a receptacle (not shown), a flexible tube 14, and a shieldable needle assembly 16. The flexible tube 14 has a first end 18 and a second end 20. The first end 18 of the flexible tube 14 is connected to the fixture 12. The first end 18 of the flexible tube 14 may be connected to the fixture 12 by means customary in the art.

The shieldable needle assembly 16 of the blood collection set 10 includes a needle cannula 22. The needle cannula 22 has a proximal end 24 and an opposing distal end 26. The needle cannula 22 defines a lumen 27 extending through the needle cannula 22 from the proximal end 24 to the distal end 26. The distal end 26 of the needle cannula 22 is beveled to define a sharp puncture tip 28, such as an intravenous (IV) puncture tip. The puncture tip 28 is provided for insertion into a patient's blood vessel, such as a vein, and is, therefore, designed to provide ease of insertion and minimal discomfort during venipuncture.

The shieldable needle assembly 16 of the blood collection set 10 includes a hub member 30, a shield member 40, and a pair of extendable members 70, 100 connecting the hub member 30 and the shield member 40. The hub member 30, shield member 40, and extendable members 70, 100 may be integrally formed as a unitary body, which is desirably molded from a thermoplastic material. Alternatively, the hub member 30, shield member 40, and extendable members 70, 100 may be separate parts, which are preferably molded from thermoplastic material.

The hub member 30 has a proximal end 32 and a distal end 34 and is generally defined as a rigid tubular wall 36 extending from the proximal end 32 to the distal end 34. The tubular wall 36 defines an internal passage 38 extending from the proximal end 32 to the distal end 34. The hub member 30 is adapted to support the proximal end 24 of the needle cannula 22. In particular, the needle cannula 22 is positioned within the internal passage 38 defined by the hub member 30 and extends outward from the distal end 34 of the hub member 30. Preferably, the needle cannula 22 and hub member 30 are formed as separate parts that are fixedly attached and secured through an appropriate medical grade adhesive, by direct mechanical attachment, or other similar means.

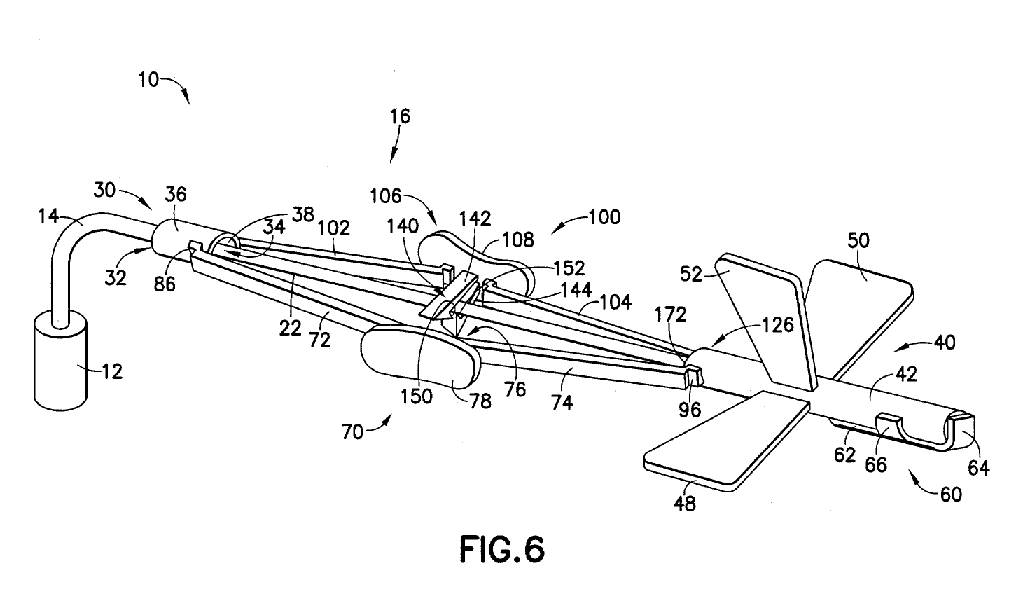

The shield member 40 and the hub member 30 are axial opposing axial directions with respect to each other between a first position in which the shield member 40 is retracted and located adjacent and, preferably, abutting the hub member 30, and a second position in which the shield member 40 is extended, such that the puncture tip 28 of the needle cannula 22 is covered by the shield member 40. In the first position, the needle assembly 16 is in a sampling state with the puncture tip 28 extending from the shield member 40 as shown in FIG. 11, and in the second position, the needle assembly 16 is in a shielded state, with the shield member 40 covering the puncture tip 28, as shown in FIG. 6.

The shield member 40 includes a housing 42 defining a central bore 44 having a distal opening 46 from which the puncture tip 28 extends when the shield member 40 is in the retracted position. The housing 42 of the shield member 40 extends co-axially about the needle cannula 22 and is in axial alignment with the internal passage 38 defined by the tubular wall 36 of the hub member 30. The shield member 40 further includes a pair of stabilizers in the form of wings 48, 50 that extend laterally from the housing 42 at opposing sides thereof. The lateral wings 48, 50 provide a butterfly-type wing assembly useful for positioning and placement of the needle assembly 16 and blood collection set 10 during a blood collection procedure. The shield member 40 may further include a dorsal wing 52 extending from the housing 42 and located between the lateral wings 48, 50. The dorsal wing 52 is preferably symmetrically positioned on the housing 42 between the lateral wings 48, 50.

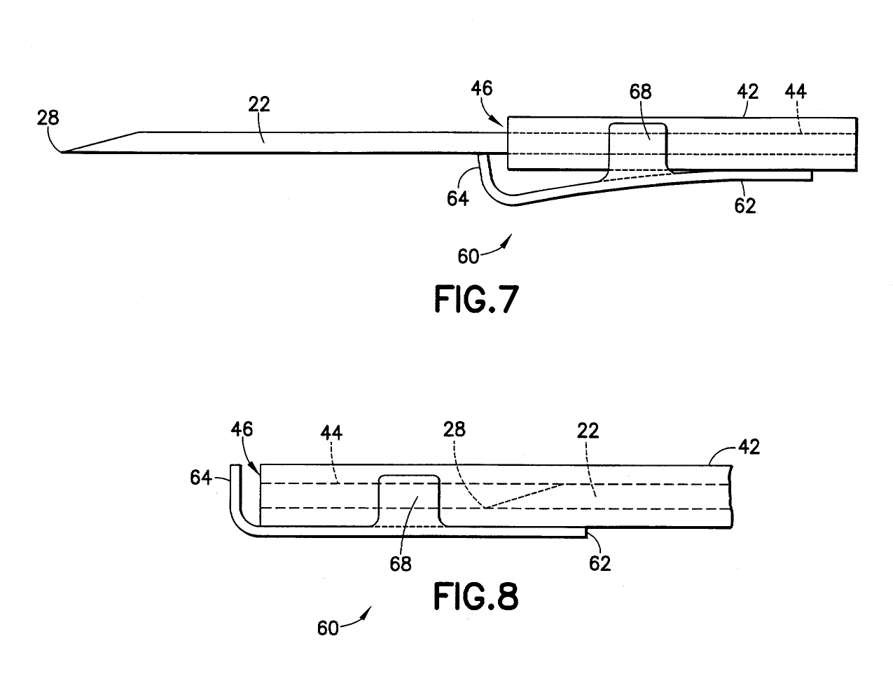

Referring now to FIGS. 1-8, a needle tip guard 60 is optionally attached to the housing 42 of the shield member 40 distally forward of the lateral wings 48, 50 and dorsal wing 52. The tip guard 60 is provided to automatically cover the distal opening 46 of the central bore 44 of the housing 42 when the shield member 40 is moved to the extended position covering the puncture tip 28 of the needle cannula 22. The tip guard 60 is provided as a curved leaf spring of metal or the like having an axially extending spring leg 62, a generally right-angled locking plate 64, and a pair of clip legs 66, 68 forming a gripping collar for securely holding the tip guard 60 onto the housing 42 of the shield member 40. Operation of the tip guard 60 shown in FIGS. 1-8 will be discussed hereinafter.

As stated previously, the hub member 30 and shield member 40 are generally connected by a pair of extendable members 70, 100. The first extendable member 70 is comprised of a pair of folding legs, which include a first or proximal leg 72 and a second or distal leg 74. The first and second legs 72, 74 are connected by a hinged knee joint 76 that includes a fingerplate 78. The first leg 72 of the pair of folding legs is hingedly connected to the hub member 30 and the second leg 74 of the pair of legs is hingedly connected to the shield member 40. The first leg 72 includes a first end 80 and a second end 82. The first end 80 of the first leg 72 is connected to the fingerplate 78 by a hinged connection 84, and the second end 82 of the first leg 72 is connected to the hub member 30 by a hub hinge at hinged connection 86. Likewise, the second leg 74 includes a first end 90 and a second end 92. The first end 90 of the second leg 74 is connected by a hinged connection 94 to the fingerplate 78, and the second end 92 of the second leg 74 is connected to the shield member 40 by a hinged connection 96. Preferably, the fingerplate 78 of the knee joint 76 is concave to provide a convenient grasping location for the user of the needle assembly 16.

Similarly, the second extendable member 100 is comprised of a pair of folding legs, which include a first or proximal leg 102 and a second or distal leg 104. The first and second legs 102, 104 are connected by a hinged knee joint 106 that includes a fingerplate 108. The first leg 102 of the pair of folding legs is hingedly connected to the hub member 30 and the second leg 104 of the pair of legs is hingedly connected to the shield member 40. The first leg 102 includes a first end 110 and a second end 112. The first end 110 of the first leg 102 is connected to the fingerplate 108 by a hinged connection 114, and the second end 112 of the first leg 102 is connected to the hub member 30 by a hub hinge at hinged connection 116. Likewise, the second leg 104 includes a first end 120 and a second end 122. The first end 120 of the second leg 104 is connected by a hinged connection 124 to the fingerplate 108, and the second end 122 of the second leg 104 is connected to the shield member 40 by a shield hinge at hinged connection 126. Preferably, the fingerplate 108 of the knee joint 106 is also concave to provide a convenient grasping location for the user of the needle assembly 16. The respective folding legs 72, 74 and 102, 104 of the first and second extendable members 70, 100 are configured to generally extend laterally along the lateral sides of the needle assembly 16 when the shield member 40 is moved to the extended position.

The needle assembly 16 further includes a locking assembly 140 located on one of the extendable members 70, 100 for locking the needle assembly 16 in the shielded state with the shield member 40 in the second extended position. In particular, the locking assembly 140 is preferably integrally formed as part of, for example, knee joint 106. The locking assembly 140 is formed by a pair of opposed locking members 142, 144. The locking members 142, 144 extend from the inside surface of fingerplate 108 and extend toward opposing fingerplate 78. The locking members 142, 144 each includes sloped leading edges 146, 148, respectively, located opposite from substantially planar locking edges 150, 152, respectively. The locking members 142, 144 define a recess therebetween configured to receive the needle cannula 22. The locking members 142, 144 are generally configured to engage the needle cannula 22 when the shield member 40 is moved to the extended position. Accordingly, the locking members 142, 144 are preferably made resiliently flexible so that the needle cannula 22 is able to separate the locking members 142, 144 as the shield member 40 is moved to the extended position. The locking assembly 140 may also be located on the fingerplate 78 of knee joint 76. The operation of the locking assembly 140 will be discussed more fully hereinafter.

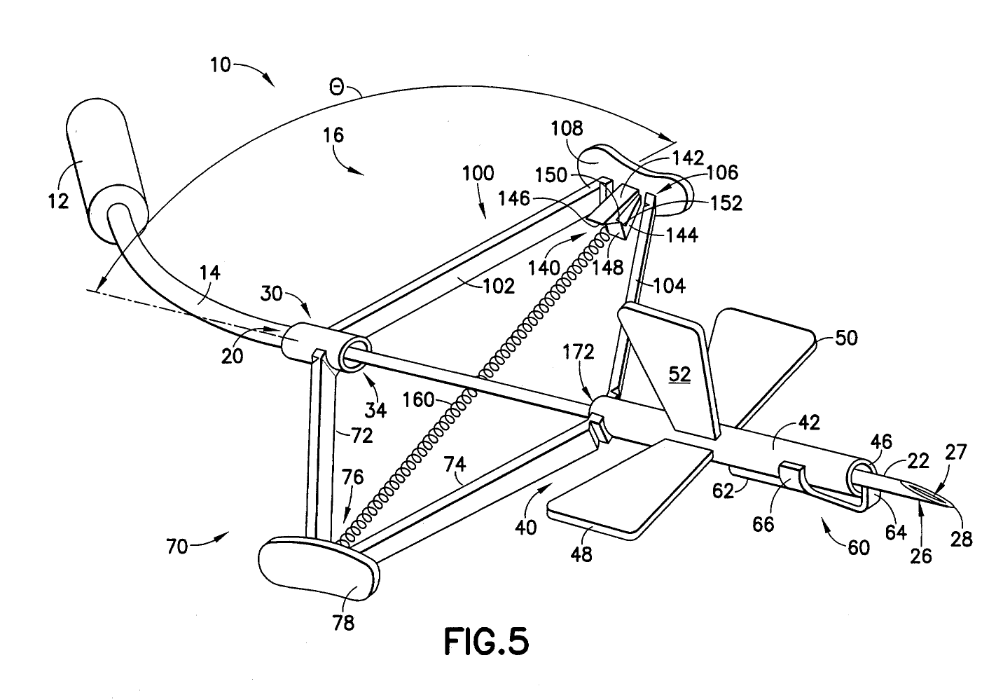

The needle assembly 16 further includes a tension spring 160 operatively connected between the fingerplates 78, 108 of the respective hinged knee joints 76, 106. In particular, the tension spring 160 is connected between inside surfaces of the fingerplates 78, 108 by means customary to the art. For example, the tension spring 160 may be mechanically attached by fasteners inside the surface of the fingerplates 78, 108 at the proximal ends of the fingerplates 78, 108. Other equivalent means may be used to attach the tension spring 160 to the fingerplates 78, 108, such as by an adhesive, and are within the scope of the present invention. The tension spring 160 is preferably adapted to provide forces that continuously bias the fingerplates 78, 108 toward one another. Accordingly, with the hub member 30 located in the position shown in FIG. 1, the tension spring 160 biases the hub member 30 into engagement with the shield member 40 through the proximal legs 72, 102 of the respective extendable members 70, 100. Once the hub member 30 is moved to a position overlapping the tension spring 160, the energy of the tension spring 160 is used to move the shield member 40 to the extended position and further draws the fingerplates 78, 108 toward one another as shown, for example, in FIG. 5 as discussed hereinafter.

A removable, protective needle cover 170 may be used to cover the distal end 26 of the needle cannula 22 and, more particularly, the puncture tip 28 of the needle cannula 22. The needle cover 170 is preferably positioned over the puncture tip 28 of the needle cannula 22 in a pre-use state of the needle assembly 16, wherein the shield member 40 is in the retracted position and maintained in the retracted position by the tension spring 160. The needle cover 170 is preferably an inexpensive, elongated plastic needle cover such as those commonly used as a needle protector in the medical field.

With the basic structure of the blood collection set 10 and needle assembly 16 described, operation of the blood collection set 10 and needle assembly 16 will be described with continued reference to FIGS. 1-8. The needle assembly 16 is preferably provided in the sampling state with the shield member 40 in the first retracted position and the needle cover 170 positioned over the distal end 26 of the needle cannula 22. The shield member 40 is held in the first retracted position by the tension spring 160. Referring to FIG. 1, the tension spring 160 biases the fingerplates 78, 108 toward one another. This force is transmitted through the proximal legs 72, 102 of the respective extendable members 70, 100 to the hub member 30. The force acting on the hub member 30 causes the hub member 30 to engage or abut the proximal end 172 of the shield member 40, with the hub hinges at hinged connections 86 and 116 positioned in front of or distal to the tension spring 160. With the shield member 40 held in the first position by the tension spring 160, a blood collection tube may be fixed to the fixture 12 located at the first end 18 of the flexible tube 14.

To use the blood collection set 10 and needle assembly 16 in a medical procedure, the user of the blood collection set 10 will first sterilize the intended area of puncture on the patient's body and remove the needle cover 170 from the distal end 26 of the needle cannula 22. The user of the needle assembly 16 may then grasp the lateral wings 48, 50 and the dorsal wing 52 to assist in positioning the needle assembly 16 at the intended area of puncture on the patient's body. The lateral wings 48, 50 and the dorsal wing 52 are preferably made flexible so that they may be folded together to provide a convenient handle for manipulating the needle assembly 16. Once the puncture tip 28 of the needle cannula 22 is inserted into a blood vessel in the patient's body (i.e., venipuncture), the user may spread the lateral wings 48, 50 flat onto the patient's body and tape them in place to maintain the positioning and placement of the needle assembly 16 during the blood collection procedure or other medical procedure. The lateral wings 48, 50 in this configuration will also provide a barrier between the needle cannula 22 and the user's fingertips, which will help prevent an accidental needle-stick should the needle cannula 22 inadvertently retract from the site of insertion. The user of the needle assembly 16 may also grasp the fingerplates 78, 108 attached to the extendable members 70, 100 with his or her free hand to further assist in positioning and placing the needle assembly 16 at the intended site of insertion into the patient's body.

After completing venipuncture, the user of the needle assembly 16 will typically collect one or more blood samples by attaching one or more blood collection tubes (not shown) to the fixture 12. Once the blood collection step is completed, the user of the blood collection set 10 and needle assembly 16 may then actuate the needle assembly 16 from the sampling state to the shielded state. To actuate the needle assembly 16, the user of the needle assembly 16 grasps the hub member 30 and begins to move the hub member 30 proximally away from the shield member 40. As the user initially moves the hub member 30 axially away from the shield member 40, the tension spring 160 continues to act upon the hub member 30 to bias the hub member 30 toward the shield member 40. The user must overcome this force to begin moving the hub member 30 away from the shield member 40.

Once the hub member 30 reaches a point when the proximal legs 72, 102 connected to the hub member 30 for each of the extendable members 70, 100 forms an angle θ of greater than 90° with the central axis on the needle cannula 22, the force of the tension spring 160 is released to automatically bias the hub member 30 and the shield member 40 away from each other in opposing axial directions to the second position. In particular the tension spring 160 is generally provided as an “over-center” spring arrangement such that when the hub member 30 is moved to a position generally overlapping the tension spring 160, the force of the tension spring 160 in released to move the fingerplates 78, 108 toward one another. The overlapping position with respect to the tension spring 160 occurs approximately when the proximal legs 72, 102 of the respective extendable members 70, 100 form the angle θ greater than 90° with the central axis of the needle cannula 22. Once the tension spring 160 is moved over-center, the tension spring 160 automatically biases the fingerplates 78, 108 toward one another, which simultaneously biases the shield member 40 and the hub member 30 axially apart. The released force of the tension spring 160 is transmitted through the proximal legs 72, 102 and the distal legs 74, 104 of the respective extendable members 70, 100 to move the shield member 40 and the hub member 30 axially apart. As the shield member 40 and the hub member 30 moves axially away from each other, the needle cannula 22 is withdrawn into the shield member 40. Thus, the tension spring 160 is used to automatically (i.e., passively), move the shield member 40 to the extended position.

The tension spring 160 may have a spring constant and free length that is sufficient to move to shield member 40 an at least partially extended position while the needle cannula 22 is in the patient's body without entirely removing the needle cannula 22 from the patient's body. For example, the tension spring 160 may be limited in strength such that as the user of the needle assembly 16 moves the hub member 30 to the position where the proximal legs 72, 102 form the angle θ of greater than 90° with the needle cannula 22, the tension spring 160 would have sufficient force to begin moving the shield member 40 to the extended position, but have insufficient force to entirely remove the needle cannula 22 from the patient's body. The user may then completely withdraw the needle cannula 22 from the patient's body. Thereafter, the tension spring 160 provides sufficient force acting on the hub and shield members 30, 40 to move the shield member 40 to the extend position covering the puncture tip 28 of the needle cannula 22.

Alternatively, the user may elect to entirely withdraw the needle cannula 22 from the patient's body before actuating the needle assembly 16. After completely withdrawing the needle cannula 22 from the patient's body, the user of the needle assembly 16 maintains the position of the shield member 40 with one hand and moves the hub member 30 axially away from the shield member 40 to the position where the proximal legs 72, 102 of the extendable members 70, 100 form an angle θ of greater than 90° with the needle cannula 22, which releases the tension spring 160 to bias the fingerplates 78, 108 toward one another and move the shield member 40 to the extended position.

In either method of operation discussed hereinabove, as the shield member 40 moves forward toward the second extended position, the locking members 142, 144 extending from fingerplate 108 of extendable member 100 move toward the needle cannula 22. As the shield member 40 travels along the needle cannula 22 toward the extended position, the sloped leading edges 146, 148 of the locking members 142, 144 slidably engage onto the needle cannula 22. As the shield member 40 reaches the fully extended position, the locking edges 150, 152 of the locking members 142, 144 snap into engagement onto the needle cannula 22 to secure the shield member 40 in the extended position. Once the locking members 142, 144 are engaged onto the needle cannula 22, the extendable members 70, 100 will be prevented from moving transversely away from the lateral sides of the needle. assembly 16. The tension spring 160 further prevents the extendable members 70, 100 from moving away from each other. Thus, the locking assembly 140, once engaged, prevents the re-emergence of the puncture tip 28 of the needle cannula 22 from the shield member 40.

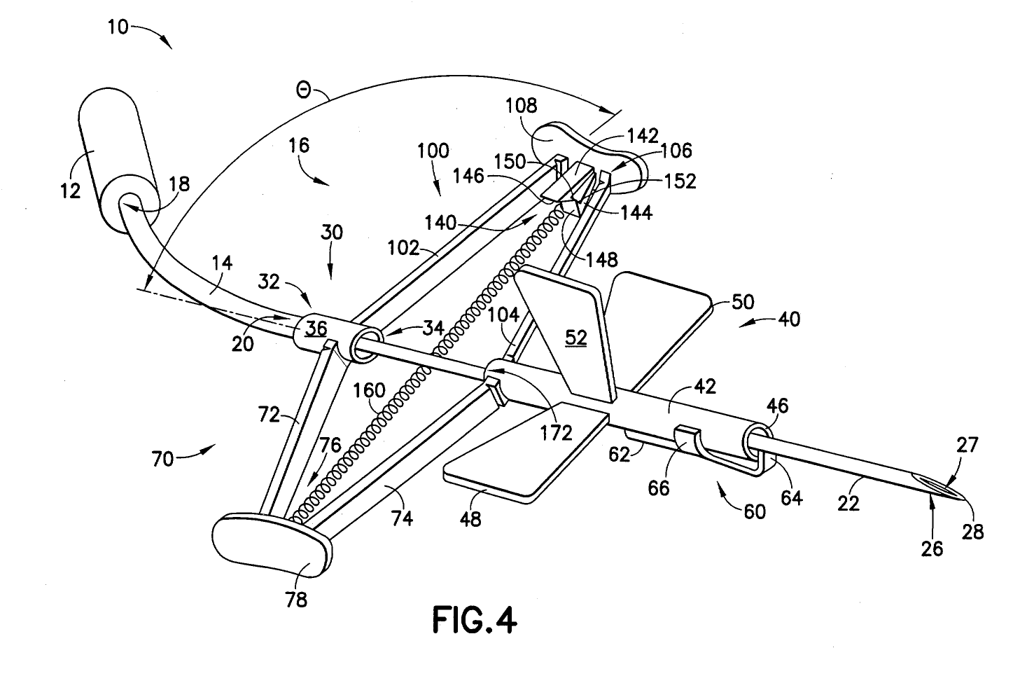

The tip guard 60 attached to the housing 42 of the shield member 40 will automatically cover the distal opening 46 of the central bore 44 once the needle cannula 22 is fully covered by the shield member 40. In particular, when the shield member 40 is in the retracted position (as in FIG. 1), the needle cannula 22 extends outward from the housing 42 of the shield member 40 and the locking plate 64 of the tip guard 60 is biased into engagement with the bottom surface of the needle cannula 22. The spring leg 62 biases the locking plate 64 into engagement with the needle cannula 22. As the needle cannula 22 begins to be covered by the shield member 40, as shown in FIGS. 4 and 5, the locking plate 64 slides along the bottom surface of the needle cannula 22 until the needle cannula 22 is fully covered by the shield member 40. Once the locking plate 64 is no longer in engagement with the needle cannula 22, the axially extending spring leg 62 causes the locking plate 64 to automatically extend over the distal opening 46 of the central bore 44 of the housing 42, as shown in FIG. 6. The tip guard 60 fully covers the distal opening 46 of the central bore 44 of the housing 42, thereby preventing any reemergence of the needle cannula 22. With the blood collection set 10 and needle assembly 16 now placed in a safety state, a blood collection tube (not shown) may be safely removed from the needle assembly 16, and the needle assembly 16 disposed of as medical waste.

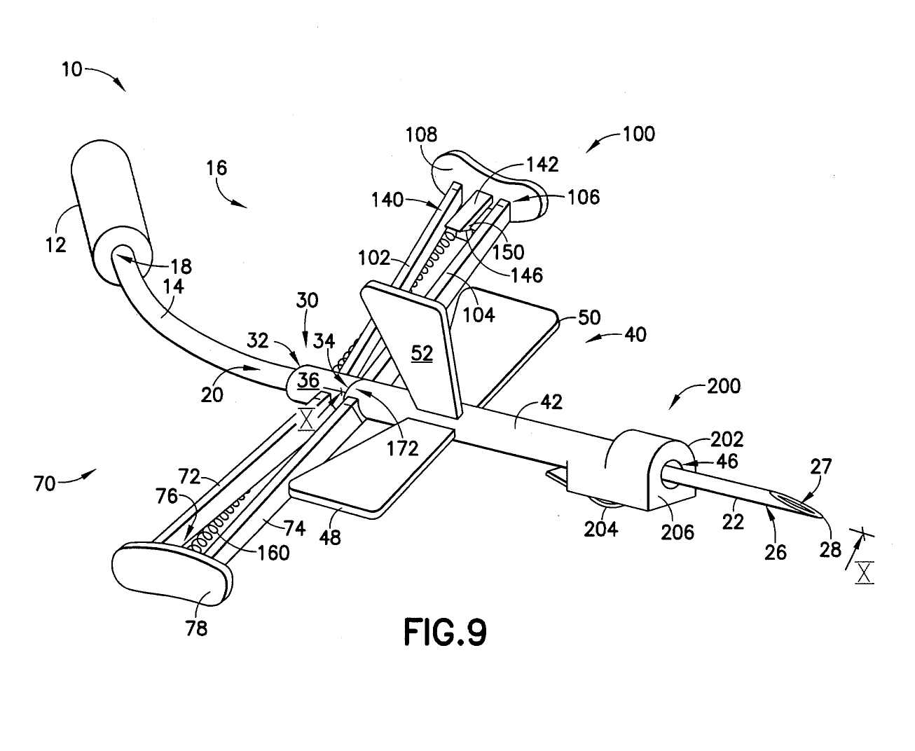

Referring to FIGS. 9-11, a further modification to the blood collection set 10 and needle assembly 16 of FIGS. 1-6 is shown. The blood collection set 10 and needle assembly 16 of FIGS. 9-11 are substantially similar to the blood collection set 10 and needle assembly 16 of FIGS. 1-6, but further include a modified needle tip guard 200. The tip guard 200 is generally comprised of a tip guard housing 202 and a protective clip 204. The tip guard housing 202 is preferably a unitary structure molded from a thermoplastic material. The tip guard housing 202 may be formed integrally with the housing 42 of the shield member 40 as shown in FIGS. 9-11, or formed separately from and attached to the housing 42 of the shield member 40. The tip guard housing 202 includes a distal end 206 and an internal passage 208 extending through the tip guard housing 202 to cooperate with the central bore 44 of the shield member 40. Portions of the internal passage 208 adjacent the distal end 206 define an enlarged clip receptacle or recess 210. A clip mounting post 212 extends downward from the tip guard housing 202. The protective clip 204 is preferably unitarily stamped and formed from a resiliently deflectable metallic material. The protective clip 204 includes a spring leg 214 with a proximal end 216 and an opposed distal end 218. A mounting aperture (not shown) extends through the spring leg 214 at a location near the proximal end 216. The mounting aperture has a diameter approximately equal to or slightly less than the diameter of the mounting post 212 of the tip guard housing 202. As such, the mounting post 212 can be forced through the mounting aperture when the axis of the mounting post 212 and the axis of the mounting aperture are substantially co-linear. A lockout leg 220 extends angularly from the distal end 218 of the spring leg 214. The lockout leg 220 is bent back toward the proximal end 216 of the spring leg 214.

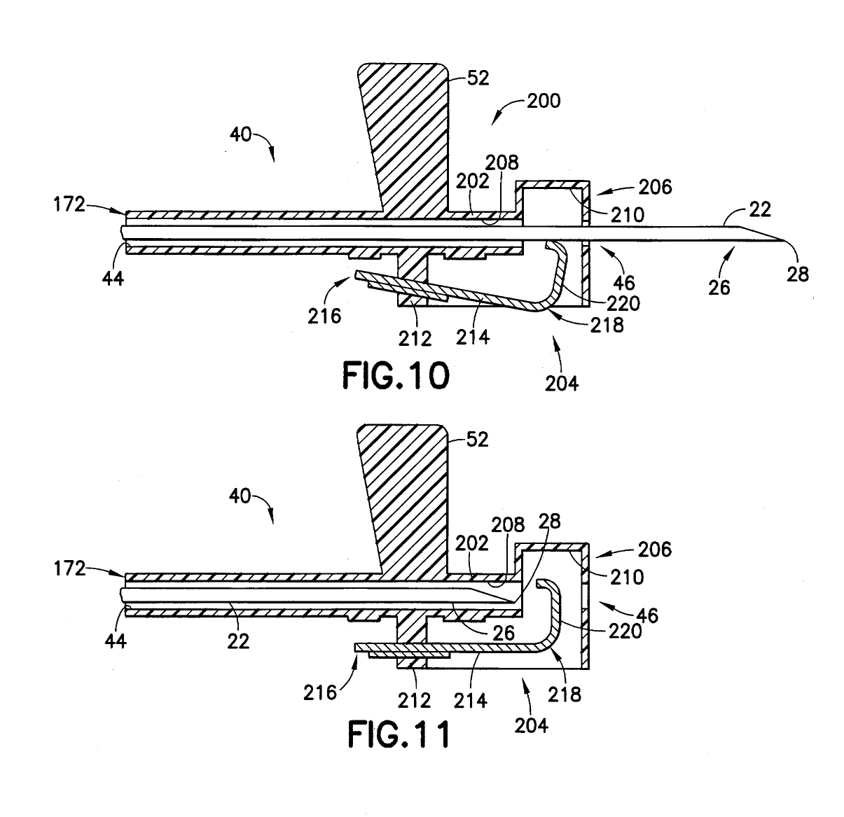

The modified tip guard 200 operates as follows. When the shield member 40 is in the retracted position, the lockout leg 220 is biased against the needle cannula 22 by the spring leg 214, as shown in FIG. 10. When the hub member 30 is moved to the position where the proximal legs 72, 102 of the extendable members 70, 100 form the angle θ of greater than 90° with the needle cannula 22, the tension spring 160 automatically (i.e., passively) moves the shield member 40 axially away from the hub member 30 to the extended position. Simultaneously, the needle cannula 22 is withdrawn into the housing 42 of the shield member 40. During the movement of the shield member 40 axially away from the hub member 30, the lockout leg 220 remains biased in contact with the needle cannula 22 by the spring leg 214. As the shield member 40 reaches its fully extended position, the needle cannula 22 withdraws axially past the recess 210 formed by the tip guard housing 202. Because the lockout leg 220 is spring-biased toward the recess 210 by the spring leg 214, as soon as the needle cannula 22 is withdrawn past the lockout leg 220, the lockout leg 220 is spring-biased into the recess 210. The lockout leg 220 thereafter prevents re-emergence of the needle cannula 22 from the tip guard housing 202 of the needle point guard 200.

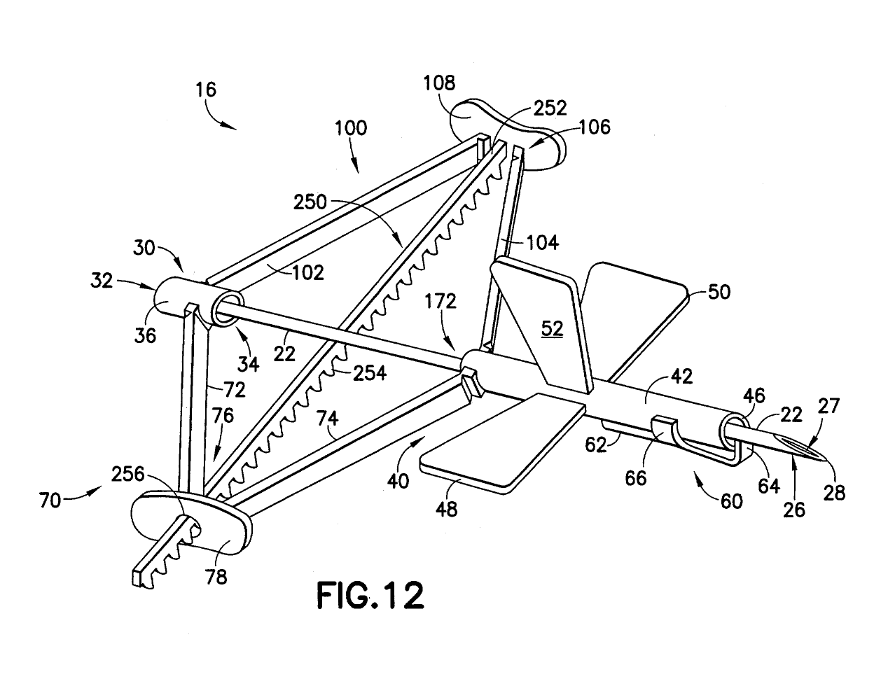

A modification to the blood collection set 10 and needle assembly 16 of FIGS. 1-6 is shown in FIG. 12. The blood collection set 10 and needle assembly 16 of FIG. 12 are substantially similar to the blood collection set 10 and needle assembly 16 of FIGS. 1-6, but no longer require the tension spring 160. Because the tension spring 160 is omitted in the blood collection set 10 and needle assembly 16 of FIG. 12, the needle assembly 16 must be manually actuated to move the shield member 40 to the extended position. This is accompanied by pressing on the fingerplates 78, 108 of the respective hinged knee joints 76, 106, which will transfer this applied force to the extendable members 70, 100. The extendable members 70, 100 through the proximal legs 72, 102 and distal legs 74, 104 will urge the hub and shield members 30, 40 apart. The shield member 40 will ultimately reach the extended position as shown in FIG. 6, discussed previously.

The blood collection set 10 and needle assembly 16 of FIG. 12 differ from the blood collection set 10 and needle assembly 16 discussed previously in that a modified locking assembly 250 is provided. The modified locking assembly 250 is comprised of a locking member 252 having a plurality of ratchet teeth 254 depending therefrom. The locking member 252 extends between the knee joints 76, 106 of the extendable members 70, 100. In particular, the locking member 252 extends from one of the fingerplates 78, 108 to the opposing fingerplates 78, 108. In FIG. 12, the locking member 252 extends from fingerplate 108 of extendable member 100 to the opposing fingerplate 78 of extendable member 70, but this configuration may be reversed. The locking member 252 extends through an opening or aperture 256 formed in fingerplate 78. The ratchet teeth 254 are preferably configured such that as inward acting force is applied to the respective fingerplates 78, 108, the ratchet teeth 254 permit the opposing fingerplates 78, 108 to move uni-directionally toward one another. As the fingerplates 78, 108 are moved toward one another, the extendable members 70, 100, through their respective proximal legs 72, 102 and distal legs 74, 104, move the shield member 40 and the hub member 30 apart. The ratchet teeth 254 are preferably configured to prevent the locking member 252 from withdrawing from the aperture 256 (i.e., move bi-directionally) in fingerplate 78, which prevents the shield member 40 from retracting axially toward the hub member 30. Thus, the locking member 252 and the ratchet teeth 254 permit one-directional movement only. The embodiment of the present invention shown in FIG. 12 may include the needle tip guard 60 or the modified needle tip guard 200, both of which were discussed previously. All other aspects of the blood collection set 10 and needle assembly 16 in FIG. 12 are substanitally similar to the blood collection set 10 and needle assembly 16 shown in FIGS. 1-6.

While the blood collection set and shieldable needle assembly of the present invention have been described with respect to preferred embodiments, various modifications and alterations of the present invention may be made without departing from the spirit and scope of the present invention. The scope of the present invention is defined in the appended claims and equivalents thereto.

What is claimed is:

1. A shieldable needle assembly, comprising:
a needle cannula having a proximal end and a distal end with a puncture tip;
a hub member supporting the proximal end of the needle cannula;
a shield member including a housing defining a central bore, the shield member in axial alignment with the hub member and extending co-axially about the needle cannula;
a pair of extendable members connecting the hub member and the shield member, with the extendable members each including a pair of folding legs connected by a hinged knee joint, and with one leg of the pair of legs for each extendable member hingedly connected to the hub member at a hub hinge and the other leg of the pair of legs for each extendable member hingedly connected to the shield member; and
a tension spring operatively connected between the extendable members,
wherein the hub member and the shield member are adapted for relative axial movement with respect to each other in opposing axial directions between a first position in which the puncture tip of the needle cannula is exposed and a second position in which the shield member covers the puncture tip of the needle cannula; wherein the hub hinge is located in front of the tension spring when the hub member and the shield member are in the first position, and wherein when the hub member is moved in an axial direction away from a proximal end of the shield, the legs connected to the hub member for each extendable member are pivoted to a position forming an angle of greater than 90° with the axis of the needle cannula, thereby causing the shield member and the hub member to be biased in opposing axial directions to the second position by the tension spring.

2. A needle assembly as in claim 1, wherein the shield member includes a pair of butterfly wings extending laterally from opposing sides of the housing of the shield member.

3. A needle assembly as in claim 2, wherein the housing of the shield member further includes a dorsal wing located between the butterfly wings.

4. A needle assembly as in claim 1, wherein the shield member includes a tip guard for protectively surrounding the puncture tip of the needle cannula when the shield member is moved to the second position, and wherein the tip guard comprises a tip guard housing and a spring clip connected to the tip guard housing, with the spring clip biased against the needle cannula when the shield member is in the first position and resiliently extends over the puncture tip of the needle cannula when the shield member is in the second position.

5. A needle assembly as in claim 1, further including a tip guard attached to the housing of the shield member and comprising a spring leg extending axially along the housing, a pair of clip legs securing the tip guard to the housing, and a locking plate resiliently biased against the needle cannula when the shield member is in the first position and resiliently extends over a distal opening to the central bore of the housing when the shield member is moved in the second position.

6. A needle assembly as in claim 1, wherein the knee joint for each extendable member further includes a fingerplate to enable the user to manipulate the assembly.

7. A needle assembly as in claim 1, further comprising a locking assembly extending from the fingerplate of at least one of the extendable members toward the needle cannula and configured to engage the needle cannula such that when the shield member and the hub member are moved in opposing axial directions to the second posotion by the tension spring, the locking member automatically engages onto the needle cannula to secure the shield member in the second position.

8. A needle assembly as in claim 7, wherein the locking assembly is comprised of a pair of opposed locking members defining a recess therebetween for receiving the needle cannula.

9. A needle assembly as in claim 8, wherein the locking members are made of a resiliently flexible material such that the needle cannula is able to separate the locking members under the force provided by the tension spring.

10. A needle assembly as in claim 1, wherein the hub member is adapted for connection to a flexible tube of a blood collection set.

11. A needle assembly as in claim 1, further including a removable protective cover positioned over the distal end of the needle cannula.

12. A shieldable blood collection set, comprising:
a flexible tube having opposed first and second ends, with the first end of the flexible tube adapted for connection to a receptacle;
a hub member connected to the second end of the flexible tube;
a needle cannula having a proximal end and a distal end with a puncture tip, the proximal end connected to the hub member and the distal end projecting from the hub member, the needle cannula defining a lumen in fluid communication with the flexible tube and the fixture;
a shield member including a housing defining a central bore, the shield member in axial alignment with the hub member and extending co-axially about the needle cannula;
a pair of extendable members connecting the hub member and the shield member, the extendable members each including a pair of folding legs connected by a hinged knee joint, and with one leg of the pair of legs for each extendable member connected hingedly to the hub member at the hub hinge and the other leg of the pair of legs for each extendable member connected hingedly to the shield member; and
a tension spring operatively connected between the extendable members,
wherein the hub member and the shield member are adapted for relative axial movement with respect to each other in opposing axial directions between a first position in which the puncture tip of the needle cannula is exposed and an a second position in which the shield member covers the puncture tip of the needle cannula, wherein the hub hinge is located in front of the tension spring when the hub member and the shield member are in the first position, and wherein when the hub member is moved in an axial direction away from a proximal end of the shield member, the legs connected to the hub member for each extendable member are pivoted to a position forming an angle of greater than 90° with the axis of the needle cannula, thereby causing the shield member and the hub member to be biased in opposing axial directions to the second position by the tension spring.

13. A shieldable blood collection set as in claim 12, wherein the shield member includes a pair of butterfly wings extending laterally from opposing sides of the housing of the shield member.

14. A shieldable blood collection set as in claim 1, wherein the shield member further includes a dorsal wing located between the butterfly wings.

15. A shieldable blood collection set as in claim 12, wherein the shield member includes a tip guard for protectively surrounding the puncture tip of the needle cannula when the shield member is in the second position, and wherein the tip guard comprises a tip guard housing and a spring clip connected to the tip guard housing, with the spring clip biased against the needle cannula when the shield member is in the first position and resiliently extends in the second to the extended position.

16. A needle assembly as in claim 1, further including a tip guard attached to the housing of the shield member and comprising a spring leg extending axially along the housing, a pair of clip legs securing the tip guard to the housing, and a locking plate resiliently biased against the needle cannula when the shield member is in the retracted position and resiliently extends over a distal opening to the central bore of the housing when the shield member is in the second position.

17. A shieldable blood collection set as in claim 12, wherein the knee joint for each extendable member further includes a fingerplate to enable the user to manipulate the needle assembly.

18. A shieldable blood collection set as in claim 12, further comprising a locking assembly extending from the fingerplate of at least one of the extendable members toward the needle cannula and configured to engage the needle cannula such that when the shield member and the hub member are moved in opposing axial directions to the second position by the tension spring, the locking assembly automatically engages onto the needle cannula to secure the shield member in the second position.

19. A shieldable blood collection set as in claim 18, wherein the locking assembly is comprised of a pair of opposed locking members defining a recess therebetween for receiving the needle cannula.

20. A shieldable blood collection set as in claim 19, wherein the locking members are made of a resiliently flexible material such that the shaft of the needle cannula is able to separate the locking members under the force provided by the tension spring.

21. A shieldable blood collection set as in claim 12, further including a removable protective cover positioned over the distal end of the needle cannula.

22. A method of automatically placing a shieldable needle assembly into a safety state in which a puncture tip of a needle cannula is covered by the assembly, comprising the steps of:
providing a shieldable needle assembly, comprising:
a needle cannula having a proximal end and a distal end with a puncture tip;
a hub member supporting the proximal end of the needle cannula;
a shield member including a housing defining a central bore, with the shield member in axial alignment with the hub member and extending co-axially about the needle cannula, and with the shield member and the hub member axially movable with respect to each other between a first position in which the puncture tip of the needle cannula is exposed and an a second position in which the shield member covers the puncture tip of the needle cannula;
a pair of extendable members connecting the hub member and the shield member, with the extendable members each including a pair of folding legs connected by a hinged knee joint, and with one leg of the pair of legs for each extendable member hingedly connected to the hub member at a hub hinge and the other leg of the pair of legs for each extendable member hingedly connected to the shield member; and
a tension spring operatively connected between the extendable members for moving the shield member and the hub member in opposing axial directions, to the extended position,
wherein the hub hinge is located in front of the tension spring when the hub member and the shield member are in the first position;
using the shieldable needle assembly in a medical procedure; and
moving the hub member in an axial direction away from a proximal end of the shield member until the legs connected to the hub member for each extendable member are pivoted to a position forming an angle of greater than 90° with the axis of the needle cannula such that the shield member and the hub member are biased in opposing axial directions to the second position by the tension spring.

23. A method as in claim 22, wherein the knee joint for each extendable member further includes a fingerplate, and the needle assembly further comprises a locking assembly extending from the fingerplate of at least one of the extendable members toward the needle cannula, with the locking assembly having a pair of opposed locking members configured to engage the needle cannula and defining a recess therebetween for receiving the needle cannula,
wherein the method further comprises the step of engaging the locking members onto the needle cannula when the shield member is biased to the second position by the tension spring.

24. A shieldable needle assembly, comprising:
a needle cannula having a proximal end and a distal end with a puncture tip;
a hub member fixedly supporting the proximal end of the needle cannula;
a shield member including a housing defining a central bore, with the shield member in axial alignment with the hub member and extending co-axially about the needle cannula, and with the shield member movable between a retracted position in which the puncture tip of the needle cannula is exposed and an extended position in which the shield member covers the puncture tip of the needle cannula;
a pair of opposing extendable members connecting the hub member and the shield member, with the extendable members each including a pair of folding legs connected by a hinged knee joint, and with one leg of the pair of legs for each extendable member hingedly connected to the hub member at a hub hinge and the other leg of the pair of legs for each extendable member connected hingedly to the shield member; and
a locking assembly connecting the knee joints of the extendable members and comprised of a locking member having a plurality of ratchet teeth, with the ratchet teeth of the locking member configured to permit uni-directional movement of the knee joints of the extendable members toward one another.

25. The shieldable needle assembly of claim 24, wherein the knee joint for each extendable member further includes a fingerplate, and wherein the locking member extends from one fingerplate to the fingerplate of the knee joint of the opposing extendable member.
